(19) United States
(12) Patent Application Publication
(10) Pub. No.: US 2008/0207952 A1
(43) Pub. Date: Aug. 28, 2008

(54) NOVEL THERAPEUTIC APPLICATIONS OF NATURAL POLY ISOPRENYLATED BENZOPHENONE DERIVATIVES AS DRUGS/MEDICAMENTS

(76) Inventor: Muhammed Majeed, Piscataway, NJ (US)

Correspondence Address: SABINSA CORPORATION, 70 ETHEL ROAD WEST, UNIT 6, PISCATAWAY, NJ, 08854, US

(21) Appl. No.: 11/858,119

(22) Filed: Feb. 18, 2008

Related U.S. Application Data
(63) Continuation-in-part of application No. 10/605,577, filed on Oct. 9, 2003.

(51) Int. Cl. C07C 49/248

(52) U.S. Cl. 568/326

(57) ABSTRACT

Disclosed are novel therapeutic properties including (i) anti-microbial activity towards acne causing bacteria; (ii) Strong anti-matrix metalloproteinase activity; and (iii) anti-cyclooxygenase 2 activities of naturally occurring poly isoprenylated benzophenone derivatives. Also disclosed are novel applications of naturally occurring polyisoprenylated benzophenones as drugs/medicaments for treating or preventing the pathological symptoms associated with (i) acne, boils and pimples; (ii) enhanced activity of matrix metalloproteinase enzyme; and (iii) enhanced activity of cyclooxygenase-2 enzyme.

This application is a continuation-in-part of pending U.S. patent application Ser. No. 10/605,577 filed on Oct. 9, 2003.

### BACKGROUND OF THE INVENTION

1. Field of the Invention

The present invention in general relates to the therapeutic potential of naturally occurring poly isoprenylated benzophenone derivatives. In particular, the present invention relates to novel therapeutic properties of naturally occurring poly isoprenylated benzophenones including (i) anti-microbial activity towards acne causing bacteria; (ii) Strong anti-matrix metalloproteinase activity; and (iii) anti-cyclooxygenase 2 activity and potential uses thereof as drugs/ medicaments for treating or preventing the pathological symptoms associated with (i) acne, boils and pimples; (ii) enhanced activity of matrix metalloproteinase enzyme; and (iii) enhanced activity of cyclooxygenase-2 enzyme.

2. Description of Prior Art

Naturally occurring poly isoprenylated benzophenones and applications thereof as cosmetics and therapeutics have been discussed in both patent and non-patent prior art references. Important references have been discussed in detail herein below.

U.S. Pat. No. 5,972,357 titled “Healthy foods and cosmetics”, to Yamaguchi, Fumio; Saito, Makoto; Ishikawa, Hiroharu; Kataoka, Shigehiro; Ariga, Toshiaki on Oct. 26, 1999 claims in principle “A cosmetic comprising a polyisoprenylated benzophenone derivative from a plant belonging to the Guttiferae family, wherein the concentration of the benzophenones is 0.00001% to 10% by weight.

U.S. Pat. No. 7,063,861 titled “Bioavailable composition of natural and synthetic HCA”, to Majeed, Muhammed, Badmaev, Vladimir on Jun. 20, 2006 claims in principle “a weight loss therapeutic method using garcinol in combination with hydroxycitric acid and anthocynanin.

Pan M H, Chang W L, Lin-Shiau S Y, Ho C T, Lin J K discuss “Induction of apoptosis by garcinol and curcumin through cytochrome c release and activation of caspases in human leukemia HL-60 cells in J Agric Food Chem. 2001 March;49(3):1464-74.

Yamaguchi F, Saito M, Ariga T, Yoshimura Y, Nakazawa H. discuss “Free radical scavenging activity and antiulcer activity of garcinol from Garcinia indica fruit rind” in the J Agric Food Chem. 2000 June;48(6):2320-5.

Tanaka T, Kohno H, Shimada R, Kagami S, Yamaguchi F, Kataoka S, Ariga T, Murakami A, Koshimizu K, Ohigashi H discuss “Prevention of colonic aberrant crypt foci by dietary feeding of garcinol in male F344 rats” in Carcinogenesis. 2000 June;21(6):1183-9.

Yamaguchi F, Ariga T, Yoshimura Y, Nakazawa H. discuss the “Antioxidative and anti-glycation activity of garcinol from Garcinia indica fruit rind” in the J Agric Food Chem. 2000 February;48(2):180-5.

Bakana P, Claeys M, Totte J, Pieters L A, Van Hoof L, Tamba-Vemba, Van den Berghe D A, Vlietinck A J discuss the “Structure and chemotherapeutical activity of a polyisoprenylated benzophenone from the stem bark of Garcinia huillensis” in the J Ethnopharmacol. 1987 September-October; 21(1):75-84.

Roux D, Hadi H A, Thoret S, Guenard D, Thoison O, Pais M, Sevenet T discuss the “Structure-activity relationship of polyisoprenyl benzophenones from Garcinia pyrifera on the tubulin/microtubule system” in the J Nat Prod. 2000 August; 63(8):1070-6.

Iinuma M, Tosa H, Tanaka T, Kanamaru S, Asai F, Kobayashi Y, Miyauchi K, Shimano R. discuss the “Antibacterial activity of some Garcinia benzophenone derivatives against methicillin-resistant Staphylococcus aureus” in the Biol Pharm Bull. 1996 February; 19(2):311-4.

While naturally occurring poly isoprenylated benzophenones have been extensively studied as “cosmetics”, the definition of which, would just classify them as cleansers, beautifiers, attraction promoters and agents that alter physical appearance, there still remains a strong need to extensively study the enormous therapeutic potential of these natural molecules as “drugs”, the definition of which would confer a whole new perspective to these molecules as agents intended to affect the structure or any function of the body of man or other animals. The present invention aims to throw light on novel therapeutic potentials of natural poly isoprenylated benzophenones useful in “health care”. More specifically, the present inventors have sought to identify novel therapeutic properties of naturally occurring poly isoprenylated benzophenone derivatives not reported earlier as prior art, which may be useful in the formulation of drugs/medicaments. The information would also be a useful addendum to the existing knowledge on the therapeutic properties of naturally occurring poly isoprenylated benzophenone derivatives.

It is thus the principle object of the present invention to evaluate and bring to light novel biological properties of naturally occurring poly isoprenylated benzophenones which have immense therapeutic value.

It is another object of the present invention to develop drugs/medicaments incorporating naturally occurring poly isoprenylated benzophenones as active principles, in lieu of their novel therapeutic biological properties.

The present invention fulfills these objectives and provides further related advantages.

### SUMMARY OF THE INVENTION

The present invention describes new, novel therapeutic properties including (i) anti-microbial activity towards acne causing bacteria; (ii) Strong anti- matrix metalloproteinase activity; and (iii) anti-cyclooxygenase 2 activities of naturally occurring polyisoprenylated benzophenone derivatives. Also described are subsequent, potential uses of naturally occurring polyisoprenylated benzophenones exhibiting novel therapeutic properties as drugs and medicaments for the pathological symptoms associated with (i) acne, boils and pimples; (ii) abnormal expression and activity of matrix metalloproteinase; and (iii) abnormal expression and activity of cyclooxygenase-2 enzyme.

The present invention provides the following advantageous features.
a) A new use of known naturally occurring polyprenylated benzophenone derivatives as therapeutic agents for the prevention/treatment/amelioration/cure/mitigation of pathological symptoms associated with acne, boils and pimples.
b) A new use of known naturally occurring polyprenylated benzophenone derivatives useful as therapeutic agents for the prevention/treatment/amelioration/cure/mitigation of pathological symptoms associated with acne, boils and pimples through the exertion of an anti-microbial effect towards acne causing bacteria.
c) A new use of known naturally occurring polyprenylated benzophenone derivatives useful as therapeutic agents for the prevention/treatment/amelioration/cure/mitigation of pathological symptoms associated with acne, boils and pimples through the exertion of an anti-microbial effect towards Propionibacterium acnes.
d) A new use of known naturally occurring polyprenylated benzophenone derivatives useful as therapeutic agents for the prevention/treatment/amelioration/cure/mitigation of pathological symptoms associated with abnormal or over-expression and activities of matrix metalloproteinase enzymes.
e) A new use of known naturally occurring polyprenylated benzophenone derivatives useful as therapeutic agents for the prevention/treatment/amelioration/cure/mitigation of pathological symptoms associated with abnormal or over-expression and activities of cyclooxygenase-2 (COX-2) enzyme.
f) Drug compositions/medicaments comprising effective concentrations of naturally occurring polyprenylated benzophenone derivatives useful as therapeutic agents for the prevention/treatment/amelioration/cure/mitigation of pathological symptoms associated with acne, boils and pimples.
g) Drug compositions/medicaments comprising effective concentrations of naturally occurring polyprenylated benzophenone derivatives useful as therapeutic agents for the prevention/treatment/amelioration/cure/mitigation of pathological symptoms associated with acne, boils and pimples through an anti-microbial effect towards acne causing bacteria.
h) Drug compositions/medicaments comprising effective concentrations of naturally occurring polyprenylated benzophenone derivatives useful as therapeutic agents for the prevention/treatment/amelioration/cure/mitigation of pathological symptoms associated with acne, boils and pimples through an anti-microbial effect towards Propionibacterium acnes.
i) Drug compositions/medicaments comprising effective concentrations of naturally occurring polyprenylated benzophenone derivatives useful as therapeutic agents for the prevention/treatment/amelioration/cure/mitigation of pathological symptoms associated with abnormal or over-expression and activities of matrix metalloproteinase enzymes.
j) Drug compositions/medicaments comprising effective concentrations of naturally occurring polyprenylated benzophenone derivatives useful as therapeutic agents for the prevention/treatment/amelioration/cure/mitigation of pathological symptoms associated with abnormal or over-expression and activities of cyclooxygenase-2 (COX-2) enzyme.
k) Drug compositions/medicaments comprising effective concentrations of naturally occurring polyprenylated benzophenone derivatives useful as therapeutic agents which offer tremendous potential towards “health care”, in lieu of their newly evaluated novel biological properties.

Other features and advantages of the present invention will become apparent from the following more detailed description, taken in conjunction with the accompanying figures, which illustrate, by way of example, the principle of the invention.

### DETAILED DESCRIPTION OF THE DRAWINGS

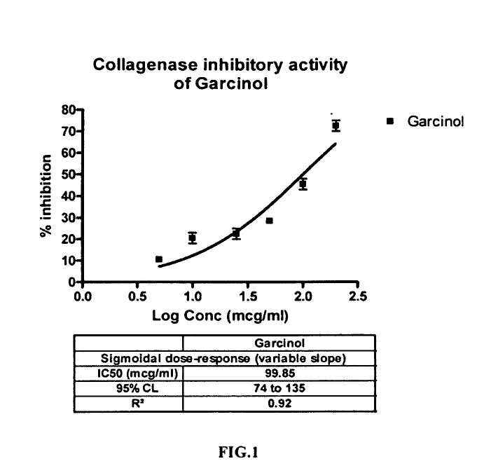

FIG. 1 shows a graphical representation of the anti-collagenase (matrix metalloproteinase enzyme) of naturally occurring poly isoprenylated benzophenones.

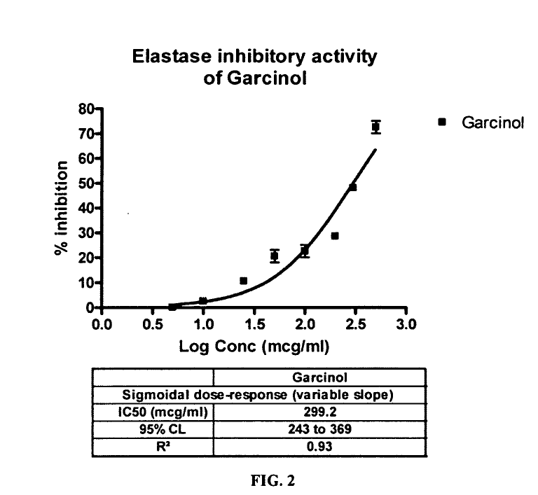

FIG. 2 shows a graphical representation of the anti-elastase (matrix metalloproteinase enzyme) of naturally occurring poly isoprenylated benzophenones.

### DETAILED DESCRIPTION OF THE PREFERRED EMBODIMENT

### Definitions

Definitions of important terms are included herein below to facilitate better understanding of the purpose and the scope of the present invention.
a) Cosmetic—SEC. 201. [21 U.S.C. 321] (i), Chapter II of the Federal Food, Drug, and Cosmetic Act (U.S. Food and Drug Administration) defines “Cosmetic” as (1) articles intended to be rubbed, poured, sprinkled, or sprayed on, introduced into, or otherwise applied to the human body or any part thereof for cleansing, beautifying, promoting attractiveness, or altering the appearance, and (2) articles intended for use as a component of any such articles; except that such term shall not include soap.
b) Drug—SEC. 201. [21 U.S.C. 321] (g) (1), Chapter II of the Federal Food, Drug, and Cosmetic Act (U.S. Food and Drug Administration) defines “Drugs” by their intended use, as (A) articles recognized in the official United States Pharmacopeia, official Homeopathic Pharmacopeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals; and (D) articles intended for use as a component of any articles specified in clause (A), (B), or (C). A food or dietary supplement for which a claim, subject to sections 403(r)(1)(B) and 403(r)(3) or sections 403(r)(1)(B) and 403(r)(5)(D), is made in accordance with the requirements of section 403(r) is not a drug solely because the label or the labeling contains such a claim. A food, dietary ingredient, or dietary supplement for which a truthful and not misleading statement is made in accordance with section 403(r)(6) is not a drug under clause (C) solely because the label or the labeling contains such a statement.
c) Acne—A chronic disorder characterized by excess production of oil from sebaceous glands causing of the hair follicles to become plugged. Pimples, papules, pustules and comedone, (black heads and white heads) cysts, infected abscesses, and sometimes scarring are the lesions to be treated in acne.
d) Matrix Metalloproteinases—A member of a group of enzymes that can break down proteins, such as collagen, that are normally found in the spaces between cells in tissues (i.e., extracellular matrix proteins). Because these enzymes need zinc or calcium atoms to work properly, they are called metalloproteinases. Matrix metalloproteinases are involved in wound healing, angiogenesis, and tumor cell metastasis.
e) Cyclooxygenases—Cyclooxygenase (COX) catalyzes the initial step of arachidonic acid metabolism and prostaglandin production. COX activity has been found to be associated with two distinct isoenzymes, COX-1 and COX-2. COX-1 is hypothesized to be involved in the maintenance of physiologic functions such as gastric protection and hemostasis, whereas COX-2 is thought to be involved in patho-physiologic processes such as inflammation, pain, and fever.

In the most preferred embodiments, the present invention relates to new therapeutic uses of naturally occurring poly isoprenylated benzophenone derivatives. In particular, the present invention describes
a) A new use of known naturally occurring polyprenylated benzophenone derivatives as therapeutic agents for the prevention/treatment/amelioration/cure/mitigation of pathological symptoms associated with acne, boils and pimples through the exertion of an anti-microbial effect towards acne causing bacteria. More preferably, the acne causing bacteria is the anaerobic bacterium Propionibacterium acnes;
b) A new use of known naturally occurring polyprenylated benzophenone derivatives useful as therapeutic agents for the prevention/treatment/amelioration/cure/mitigation of pathological symptoms associated with the over-expression and hyper-activities of matrix metalloproteinase enzymes;
c) A new use of known naturally occurring polyprenylated benzophenone derivatives useful as therapeutic agents for the prevention/treatment/amelioration/cure/mitigation of pathological symptoms associated with the over-expression and hyper-activities of cyclooxygenase-2 (COX-2) enzyme.

In additional embodiments, the present invention also describes
a) Drug compositions/medicaments comprising effective concentrations of naturally occurring polyprenylated benzophenone derivatives useful as therapeutic agents for the prevention/treatment/amelioration/cure/mitigation of pathological symptoms associated with acne, boils and pimples. In a preferred embodiment, the said drug compositions/medicaments exert an anti-microbial effect towards acne causing bacteria. More preferably, the said drug compositions/medicaments exert an anti-microbial effect towards anaerobic bacterium Propionibacterium acnes;
b) Drug compositions/medicaments comprising effective concentrations of naturally occurring polyprenylated benzophenone derivatives useful as therapeutic agents for the prevention/treatment/amelioration/cure/mitigation of pathological symptoms associated with the over-expression and hyper-activities of matrix metalloproteinase enzymes;
c) Drug compositions/medicaments comprising effective concentrations of naturally occurring polyprenylated benzophenone derivatives useful as therapeutic agents for the prevention/treatment/amelioration/cure/mitigation of pathological symptoms associated with the over-expression and hyper-activities of cyclooxygenase-2 (COX-2) enzyme.
d) Drug compositions/medicaments comprising effective concentrations of naturally occurring polyprenylated benzophenone derivatives useful as therapeutic agents which offer tremendous potential towards “health care”, in lieu of their newly evaluated novel biological properties.

In preferred embodiments, the drug compositions/medicaments referred herein above have effective concentrations of naturally occurring poly isoprenylated benzophenone derivatives from about 0.001%-10% w/w. More preferably, the drug compositions/medicaments referred herein above have effective concentrations of naturally occurring poly isoprenylated benzophenone derivatives from about 0.1%-5% w/w.

The new uses of naturally occurring poly isoprenylated benzophenones as mentioned herein above have been deduced from a careful evaluation of novel biological properties of the said molecules. The experimental evaluation of the aforesaid biological properties and the associated results has been mentioned herein below as specific examples.

### EXAMPLE I

Anti-Microbial Activity Against Propionibacterium acnes

Propionibacterium acnes was cultured in an anaerobic chamber (Coy Laboratory, USA with an automatic lock purge system). The growth standardization was ensured by culturing the organism in different culture media. The anaerobic conditions were ensured initially with Nitrogen gas and then a mixture of Nitrogen, Hydrogen and Carbon dioxide in a ratio of 80:10:10. A two gas tank arrangement was made wherein only Nitrogen was connected to the transfer chamber and the mixed gas to the main chamber using Gassing Manifold (Hrishi Biotech. Pune, India). The anaerobic media were prepared by heating the media while passing the mixture of gases Nitrogen and Carbon dioxide in a ratio of 4:1 simultaneously. A redox indicator reazurin=0.001% was added to the media. The indicator is colorless in an anaerobic environ and turns blue otherwise. It was dispensed to 30 ml, 20 ml and 10 ml vials sealed with rubber and aluminium clamps and sterilized by autoclaving at 121° C. for 15 minutes.

Propionibacterium acnes ATCC 11827 was sub cultured from broth cultures into vials containing fresh medium. The cultures were then incubated for 48 hours at 37° C. The optical density of the culture at 625 nm was maintained between 0.64 and 0.80 which corresponded to approximately 12×10⁶ cells/ml (4.0 MC Mc Farland standard). The anti-microbial susceptibility testing was carried on Reinforced Clostridial Agar (RCA). 0.3 ml of P. acnes culture was inoculated into the culture plates. Culture plates were impregnated with disks comprising varying concentrations of the test naturally occuring poly isoprenylated benzophenone derivatives. The plates were incubated in anaerobic chambers at 37° C. for 48 hours. The test naturally occuring poly isoprenylated benzophenone derivatives were prepared with DMSO as vehicle. The controls included DMSO and Clindac A (Clindamycin Phosphate Gel 1% w/w). The zone of inhibition was measured and expressed in mm. (Table I)

TABLE I

| | Conc. | Zone of Inhibition (mm) | | | | | |
|---|---|---|---|---|---|---|---|
| | Of the | Ext | Ext | Ext | Garcinol | Cambogin | Clindac |
| S. No | sample | A | B | C | 90% | 90% | A |
| 1 | 5.0% | 7 | 8 | 10 | 20 | 0 | 20 |
| 2 | 2.0% | 0 | 7 | 8 | 19 | 0 | 16 |
| 3 | 1.0% | 0 | 0 | 8 | 18 | 0 | 7 |
| 4 | 0.5% | 0 | 0 | 7 | 16 | 0 | 0 |
| 5 | 0.3% | — | — | 0 | 14 | — | 0 |
| 6 | 0.1% | — | — | 0 | 11 | — | 0 |
| 7 | 0.05% | — | — | — | 10 | — | 0 |
| 8 | 0.03% | — | — | — | 8 | — | 0 |
| 9 | 0.01% | — | — | — | 7 | — | 0 |

Extract A-Garcinol 3% and Cambogin 2%
Extract B-Garcinol 20% and Cambogin 10%
Extract C-Garcinol 40% and Cambogin 20%

### EXAMPLE II

Anti-Collagenase (Anti-Metalloproteinase Activity) (FIG. 1 and Table II)

Principle: The substrate DQ gelatin (from pig skin), fluorescein conjugate—gelatin is efficiently digested by most of the collagenases to yield highly fluorescent peptides. The increase in fluorescence is proportional to proteolytic activity and the fluorescence is quenched in the presence of inhibitor. The reduction in fluorescence intensity was measured in a microplate reader (Emission wavelength: 485 nm and Excitation wavelength: 520 nm.)

Methodology: Varying concentrations of the test material were pre-incubated for 10 minutes with the substrate DQ gelatin. Collagenase Type IV from Clostridium histolyticum enzyme was then added and the fluorescence intensity was measured after 30 minutes (Em: 485 nm and Ex: 520 nm.).

Result:

IC₅₀ of Garcinol is 99.85 μg/ml. 95% confidence limit—74 to 135 μg/ml.

TABLE II
Garcinol Sigmoidal Dose Response Curve
for Anti-Collagenase Activity

| | |
|---|---|
| IC₅₀ (mcg/ml) | 99.85 |
| 95% CL | 74-135 |
| R² | 0.92 |

### EXAMPLE 3

Anti-Elastase (Anti-Matrix Metalloproteinase) Activity (FIG. 2, Table III)

Principle: The assays were done using the EnzChek elastase assay kit. The substrate is DQ elastin soluble bovine neck ligament. DQ elastin is labeled with BODIPY FL dye. The non-fluorescent substrate can be digested by elastase to yield highly fluorescent fragments and in the presence of inhibitor, the fluorescence intensity is quenched. The fluorescence intensity was measured in a microplate reader (emission at 485 nm and excitation at 520 nm.)

Methodology: Varying concentrations of the test material were pre-incubated for 10 minutes with the substrate DQ gelatin. Elastase from Pig pancreas enzyme was then added and the fluorescence intensity was measured after 30 minutes (Em: 485 nm and Ex: 520 nm.).

Result:

IC50 of Garcinol is 300 μg/ml

95% confidence limit—250 to 365 μg/ml

TABLE III
Garcinol Sigmoidal Dose Response
Curve for Anti-Elastase Activity

| | |
|---|---|
| IC₅₀ (mcg/ml) | 299.2 |
| 95% CL | 243-369 |
| R² | 0.93 |

### EXAMPLE 4

Principle: The COX inhibitor screening Assay was performed using the Cox inhibitory kit from Cayman chemical company (Catalog No. 560101) by Enzyme Immuno Assay method.

Methodology: Enzyme (COX-1) is allowed to react on substrate Arachidonate in presence & absence of the inhibitor. The amount of prostaglandins (PGF2

[symbol]

) produced by stannous chloride reduction of COX-derived PGFH2 are estimated by EIA (Cayman) using a broadly specific antibody that binds to all major prostaglandin compounds. The decrease in PGs released in presence of inhibitor is directly proportional to inhibition.

Result

IC50 of Garcinol for COX 1 is 87

[symbol]

g/ml. 95% Confidence limit is 80 to 90 μg/ml

IC50 of Garcinol for COX 2 is 22

[symbol]

g/ml. 95% Confidence limit is 20 to 25 μg/ml

While the invention has been described with reference to a preferred embodiment, it is to be clearly understood by those skilled in the art that the invention is not limited thereto. Rather, the scope of the invention is to be interpreted only in conjunction with the appended claims.

We claim:

1. The use of naturally occurring poly isoprenylated benzophenone derivatives in the preparation of an anti-acne medicament in the ready-to-use drug form for treating or preventing pathological symptoms associated with acne, boils and pimples.

2. The use of naturally occurring poly isoprenylated benzophenone derivatives in the preparation of an anti-acne medicament in the ready-to-use drug form for treating or preventing pathological symptoms associated with acne, boils and pimples according to claim 1, wherein the said medicament exerts an anti-microbial effect on acne causing bacteria.

3. The use of naturally occurring poly isoprenylated benzophenone derivatives in the preparation of an anti-acne medicament in the ready-to-use drug form for treating or preventing pathological symptoms associated with acne, boils and pimples according to claim 1, wherein the said medicament exerts an anti-microbial effect on acne causing Propionibacterium acnes.

4. The use of naturally occurring poly isoprenylated benzophenone derivatives in the preparation of an anti-metallo proteinase medicament in the ready-to-use drug form for treating or preventing pathologies associated with an abnormally enhanced activity of metalloproteinase enzymes.

5. The use of naturally occurring poly isoprenylated benzophenone derivatives in the preparation of an anti-cyclooxygenase 2 medicament in the ready-to-use drug form for treating or preventing pathologies associated with an abnormally enhanced activity of cyclooxygenase 2 enzymes.

6. Ready-to-use drugs/medicaments comprising effective concentrations of naturally occurring poly isoprenylated benzophenone derivatives for treating or preventing the pathological symptoms associated with acne, boils and pimples.

7. Ready-to-use drugs/medicaments comprising effective concentrations of naturally occurring poly isoprenylated benzophenone derivatives for treating or preventing the pathological symptoms associated with acne, boils and pimples, wherein the said drugs/medicaments exert an anti-microbial effect on acne causing bacteria.

8. Ready-to-use drugs/medicaments comprising effective concentrations of naturally occurring poly isoprenylated benzophenone derivatives for treating or preventing the pathological symptoms associated with acne, boils and pimples, wherein the said drugs/medicaments exert an anti-microbial effect on acne causing Propionibacterium acnes.

9. Ready-to-use drugs/medicaments comprising effective concentrations of naturally occurring poly isoprenylated benzophenone derivatives for treating or preventing pathologies associated with an enhanced activity of metalloproteinase enzymes.

10. Ready-to-use drugs/medicaments comprising effective concentrations of naturally occurring poly isoprenylated benzophenone derivatives for treating or preventing pathologies associated with an enhanced activity of cyclooxygenase 2 enzymes.

11. Ready-to-use drugs/medicaments as in one of claims 6, 7, 8, 9 or 10, in which the effective concentrations of naturally occurring poly isoprenylated benzophenone derivatives is from about 0.001 to about 10% w/w.

12. Ready-to-use drugs/medicaments as in one of claims 6, 7, 8, 9 or 10, in which the effective concentrations of naturally occurring poly isoprenylated benzophenone derivatives is from about 0.1% to about 5% w/w.
